(19) United States
(12) Patent Application Publication
(10) Pub. No.: US 2015/0355159 A1
(43) Pub. Date: Dec. 10, 2015

(54) MEASURING APPARATUS, KIT, AND METHOD OF USING SAME

(71) Applicant: John Kocak, Cliffside Park, NJ (US)

(72) Inventor: John Kocak, Cliffside Park, NJ (US)

(21) Appl. No.: 14/299,378

(22) Filed: Jun. 9, 2014

(51) Int. Cl. G01N 33/38; G01B 5/207

(52) U.S. Cl. CPC G01N 33/381; G01B 5/207

(57) ABSTRACT

A handheld digital measuring device, method and kit therefore are provided. The device includes first and second elongated, substantially flat blade members having proximal and distal ends. At a proximal end thereof, the blade members are fixed to and extend from a housing. The first and second blade members are movable relative to one another in an opposing manner to provide a defined and measurable distance between the distal ends of the first second blade members. Optionally, the device may have a photosensor for sensing a dimension of the distance between the distal ends of the blade members and a microcomputer responsive to photosensor signals for transmitting the distance to a display device. The device may also comprise a spring-loaded pneumatic mechanism for urging the blade members apart. The device is preferably handheld and includes buttons or a keypad for controlling operation thereof.

### BACKGROUND OF THE INVENTION

1. Field of the Invention

The present invention generally relates to a handheld device for measuring the dimensions of gemstones and other objects of similar size or dimensions, and, more particularly, to a measurement apparatus that is easy to use and has high resolution calibration with a pair of tweezer-like blades or jaws. Although the apparatus of the present invention is intended primarily for measurement of gemstones, it is not limited to such use. Furthermore, the apparatus is not limited to a handheld device.

2. Description of the Related Art

Gemstones such as diamonds, emeralds and rubies are classified according to weight and shape. The weight is commonly determined by measuring one to three dimensions of the diamond with a device and referring to appropriate tables for the corresponding weight. Previously the measurements would be taken with a standard set of mechanical calipers which utilize either spring-loaded caliper jaws or thumbwheel control of the caliper jaws adjacent the respective surfaces of the gemstone.

Manufacturers of jewelry incorporating gemstones have long recognized the necessity to be able to accurately determine the physical size and therefore the value of gemstones. This necessity is created by the frequent need to determine the weight of the gemstone or to properly measure dimensions for specific designs. In conventional instruments used to measure linear dimensions, a vernier scale or dial indicator is generally used. However, this category of instruments is inadequate because of the probability of misreading the typical analog display due to reading errors and human error in that the user is required to interpret mechanically measured dimensions. Such conventional measuring instruments are also inadequate in that the primary scale of measurement is based upon a fixed calibration of the instrument.

Digital measuring instruments which can be used for measuring diamonds are known. One such instrument utilizes a set of caliper jaws movable by a thumbwheel. As the caliper jaws move, a rack and pinion arrangement sends signals to a computer. The computer stores tables for estimation of the gemstone weight which is displayed on a digital display. Digital calipers are also known. One such digital caliper is a transparent linear scale having calibrations and a light emitter positioned to direct light through the scale. A light detector on the opposite side of the scale counts the calibration lines as the caliper jaws move so as to determine linear travel. Another such caliper device wherein a first rotatable member is rotated relative to a second rotatable member, and bright and dark patterns of the fringe are sensed to measure dimension.

Prior art gemstone measurement devices have various drawbacks and disadvantages. Mechanical scales and prior electronically sensed linear scales provide limited resolution, since linear scales which move the same distance as the caliper jaws cannot be expanded. Devices utilizing thumbwheels for caliper adjustment are inconvenient and relatively slow in use. Spring-loaded calipers tend to snap back to the closed position and often dislodge gemstones from the calipers. The use of tables for determining gemstone weight based on dimensional measurement is relatively inaccurate and requires inconvenient interpolation between tabulated points.

Yet another known device is a handheld digital gemstone measurement apparatus comprising a housing and a first caliper jaw fixed to the housing, a second caliper jaw linearly movable relative to the first caliper jaw, a rotary encoding wheel mounted for rotation relative to the housing and including a plurality of radial slots, means responsive to linear motion of the second caliper jaw for causing rotation of the encoding wheel, means for sensing rotation of the encoding wheel by optical sensing of the radial slots and providing a signal representative of the linear travel of the second caliper jaw to provide a dimensional measurement of a gemstone positioned between the caliper jaws, and means responsive to one or more dimensional measurements of the gemstone for calculating the weight thereof.

Such a device also comprises a housing and a first caliper jaw fixed to the housing, a second caliper jaw linearly movable relative to the first caliper jaw, means for sensing movement of the second caliper jaw and providing a signal representative of linear travel of the second caliper jaw to provide a dimensional measurement of a gemstone positioned between the caliper jaws, and means responsive to one or more dimensional measurements of the gemstone for calculating the weight thereof, including means for selecting a gemstone shape from a group of programmed shapes, means responsive to the selected gemstone shape, the one or more dimensional measurements and a predetermined gemstone density for computing the gemstone weight, and means for displaying the gemstone weight. The calculating means may include a keypad for selecting gemstone shape, a microprocessor for receiving inputs from the keypad and from the sensing means for computing the gemstone weight and for energizing the display means.

Such measuring instruments have progressed through the use of digital displays. The devices are generally referred to as caliper apparatus. A caliper apparatus disclosed by the prior art utilizes a magnescale in which magnetic calibrations are provided on a magnetic tape and a signal produced due to the movement of the scale relative to the magnetic tape. This is sensed to measure dimension. Another caliper device employs a linear encoder in which light is directed toward an optical measuring device in which light is reflected from a first member and received by a second member of the device. Reflected light is sensed to measure dimension. In yet another caliper device, a rotary encoder is employed in which a rotatable first member is rotated relative to a rotatable second member and bright and dark patterns of light are sensed to measure dimension. In all these cases, the problem related to the manual interpretation of a measurement is replaced by a digital display which eliminates reading errors and human error. However, these devices continue to be inadequate for the measurement of gemstones since the measurement of the gemstone along multiple axes has required manual intervention by the user.

Another such measuring device measures the dimensions of multiple axes of the gemstone. Although this device also provides means for automatically calculating the gemstone weight, it suffers from the same inadequacies as other devices described in the prior art. To measure multiple axes of a gemstone, these devices all require the manual manipulation of the gemstone to permit measurement of the relevant dimensions thereof. One such measuring device measures the dimensions of small objects in general, and gemstones in particular. The structural features of the device can be employed with any number of conventional measurement apparatus. For example, the device employs a measuring apparatus pursuant to which selected dimensions are measured between a pair of measurement flanges. One of the measurement flanges is fixed, the second measurement flange being moveable to receive the gemstone irrespective of size. A rotatable platform is disposed adjacent the fixed measurement flange, the axis of the rotatable platform adapted to be perpendicular to the dimension of the gemstone being measured. In operation, the gemstone is placed on the rotatable platform, the axis of the gemstone to be measured to be aligned with the axis of the aligned measurement flanges. A plurality of dimensions of the gemstone may be measured merely by altering the position of the rotatable platform about its axis. The measurement of all relevant dimensions of the gemstone may be performed without any manual contact between the user and the gemstone.

Other known instruments in the prior art which are employed for measuring the physical dimensions of gemstones exhibit additional inadequacies. Gemstones must be measured along multiple of axes in order to provide the information necessary to determine actual size and value. Such a problem is not in any way addressed by the conventional measuring instruments in the prior art.

Accordingly, this invention now recognizes that there is a need for resolving those inadequacies inherent in the devices disclosed in the prior art. The present invention employs tweezer-like blades electronically interconnected to a display device for the simple, efficient and accurate measurement of small objects, such as gemstones, pharmaceuticals, etc. The improvement provided by the present invention relates to the ability to do in one step that which has long been done in multiple steps.

### SUMMARY OF THE INVENTION

A handheld digital measuring device, method and kit therefor is disclosed. The device includes first and second elongated, substantially flat blade members having proximal and distal ends. At a proximal end thereof, the blade members are fixed to and extend from a housing. The first and second blade members are movable relative to one another in an opposing manner to provide a defined and measurable distance between the distal ends of the first second blade members. Optionally, the device may have a photosensor for sensing a dimension of the distance between the distal ends of the blade members and a microcomputer responsive to photosensor signals for transmitting the distance to a display device. The device may also comprise a spring-loaded pneumatic mechanism for urging the blade members apart. The device is preferably handheld and includes buttons or a keypad for controlling operation thereof.

According to one aspect of the invention, there is provided a handheld digital gemstone measuring device comprising a housing having first and second blade members extending therefrom, wherein the first and second blade members each having proximal and distal ends. The first blade member is preferably movable in an opposite direction relative to the second blade member such that a sensor may sense movement of the blade members relative to one another and provide a signal representative of the opposing distance between the blades members at the distal end thereof to provide a dimensional measurement of an object positioned therebetween.

The measuring device of the invention further comprises a display device for receiving the signal from the sensor in order to provide a visual display of the dimensional measurement of the object. The display device of the measuring device may further include a keypad or buttons for selecting a format for display of dimensional measurement, for resetting the display device, for calibrating the measuring device, and the like. The display device may optionally include means for resetting the dimensional measurements at any selected position along the blade members. The measuring device may also further comprise a spring-loaded pneumatic mechanism coupled between the housing and the distal end of the blade members for urging the blade members apart from one another for ease in collection of the object.

According to still another aspect of the invention, there is provided a kit for providing a handheld digital measuring device or apparatus comprising a case body having a closure mechanism, at least one handheld measuring device having a display and a housing for receiving a pair of blade members, and at least one pair of interchangeable blade members that may be interchangeably and detachably engaged with the housing. Preferably, the closure mechanism is a zipper closure, and the measuring device and blade members are removably secured to the inside of the case by elastic loops. Optionally, the blade members comprise at least one of a slant tip, blunt tip, or pointed tip tweezer.

The measurement apparatus of the present invention, although intended primarily for measurement of gemstones, can be utilized for measurement of any number of small objects, such as pharmaceuticals and the like. Furthermore, the features of the invention can be embodied in measurement apparatus which is not handheld, as well as in measurement devices that are wireless. The calculating means can be configured to determine any desired quantity based on measured dimensions.

In another aspect of the present invention, a method for measuring diamonds is provided, wherein the method comprises the steps of calibrating the measuring device, locating an object to be measured, securing the object at a distal end of a pair of blade members, and reading a displayed dimension of the object on a display device.

It is therefore an object of the present invention to provide an improved gemstone measurement apparatus.

It is also an object of the present invention to provide an improved gemstone measurement apparatus which includes an ultra violet light source for checking the fluorescence of the gemstone.

It is an object of the present invention to provide an improved gemstone measurement apparatus which includes a counting mechanism to count the gemstones measured upon touch of a pressure sensor.

It is an object of the present invention to provide an improved gemstone measurement apparatus comprised of very rigid metallic or polymer materials such that the tweezer blades are more rigid to yield more accurate measurements.

It is an object of the present invention to provide an improved multifunction gemstone measurement apparatus.

It is an object of the present invention to provide an improved gemstone measurement apparatus which includes a laser for reading and measuring the object.

It is an object of the present invention to provide an improved gemstone measurement apparatus which uses a gear mechanism to measure the object.

It is an object of the present invention to provide an improved gemstone measurement apparatus which uses a magnetic device for measuring the object.

It is an object of the present invention to provide an improved measurement apparatus which can be used to measure objects other than gemstones, including pharmaceuticals and the like.

It is another object of the present invention to provide an improved gemstone measurement apparatus which permits measurement along multiple axes of the gemstone with relative ease.

It is still yet another object of the present invention to provide an improved gemstone measurement apparatus which is simple and inexpensive to fabricate.

It is a further object of the present invention to provide improved methods and apparatus for measuring the dimensions and the weight of gemstones and other objects.

It is another object of the present invention to provide a handheld digital gemstone measurement apparatus having increased accuracy.

It is a further object of the present invention to provide a handheld digital gemstone measurement apparatus that is convenient to use.

It is still another object of the present invention to provide a handheld digital gemstone measurement apparatus which avoids the necessity for reference to weight tables and interpolation between tabulated values.

It is yet another object of the present invention to provide a handheld digital gemstone measurement apparatus which is self-contained and includes a direct readout.

It is yet another object of the present invention to provide handheld digital gemstone measurement apparatus including a magnetic means for controlling movement of tweezer blades.

The above and other aspects, features and advantages of the present invention will become apparent from the following description read in conjunction with the accompanying drawings, in which like reference numerals designate the same elements.

### BRIEF DESCRIPTION OF THE DRAWINGS

A further understanding of the present invention can be obtained by reference to a preferred embodiment set forth in the illustrations of the accompanying drawings. Although the illustrated preferred embodiment is merely exemplary of methods, structures and compositions for carrying out the present invention, both the organization and method of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the present invention, reference is now made to the following drawings in which:

FIG. 1 shows a perspective view of a measuring device in accordance with an exemplary embodiment of the present invention;

FIG. 2 shows a side view of the measuring device shown in FIG. 1, further showing a partial transparent view of a portion of the measuring device;

FIG. 3 shows a top plan view of the measuring device shown in FIG. 1;

FIG. 4 shows an end view of the measuring device of FIG. 1;

FIG. 5 shows a front elevational view of a pair of tweezer blade tips according to a first alternative embodiment of the present invention;

FIG. 6 shows a front elevational view of a pair of tweezer blade tips according to a second alternative embodiment of the present invention;

FIG. 7 shows a front elevational view of a pair of tweezer blade tips according to a third alternative embodiment of the present invention;

FIG. 8 shows a front elevational view of a pair of tweezer blade tips according to a fourth alternative embodiment of the present invention;

FIG. 9 shows a front elevational view of a pair of tweezer blade tips according to a fifth alternative embodiment of the present invention;

FIG. 10 shows a schematic diagram of exemplary circuitry for use in the handheld measurement device of the present invention;

FIG. 11 shows a flow chart illustrating an exemplary operation of the handheld measurement device of the present invention;

FIG. 12A shows a perspective view of an exemplary measurement device kit, including a case having a zippered closure, suitable for containing the tweezer measurement device with a variety of tweezer blades; and

FIG. 12B shows the kit of FIG. 12A in an open configuration.

### DETAILED DESCRIPTION OF THE PREFERRED EMBODIMENTS

As required, a detailed illustrative embodiment of the present invention is disclosed herein. However, techniques, systems, compositions and operating structures in accordance with the present invention may be embodied in a wide variety of sizes, shapes, forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein which define the scope of the present invention.

Reference will now be made in detail to several embodiments of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as top, bottom, up, down, over, above, below, etc., or motional terms, such as forward, back, sideways, transverse, etc. may be used with respect to the drawings. These and similar directional terms should not be construed to limit the scope of the invention in any manner.

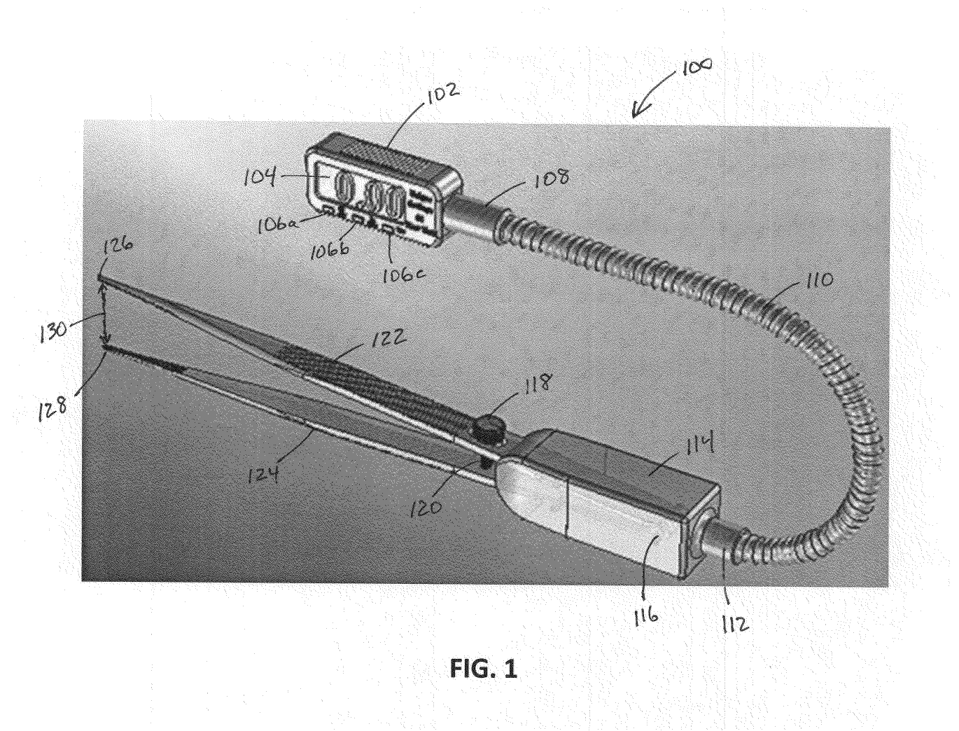

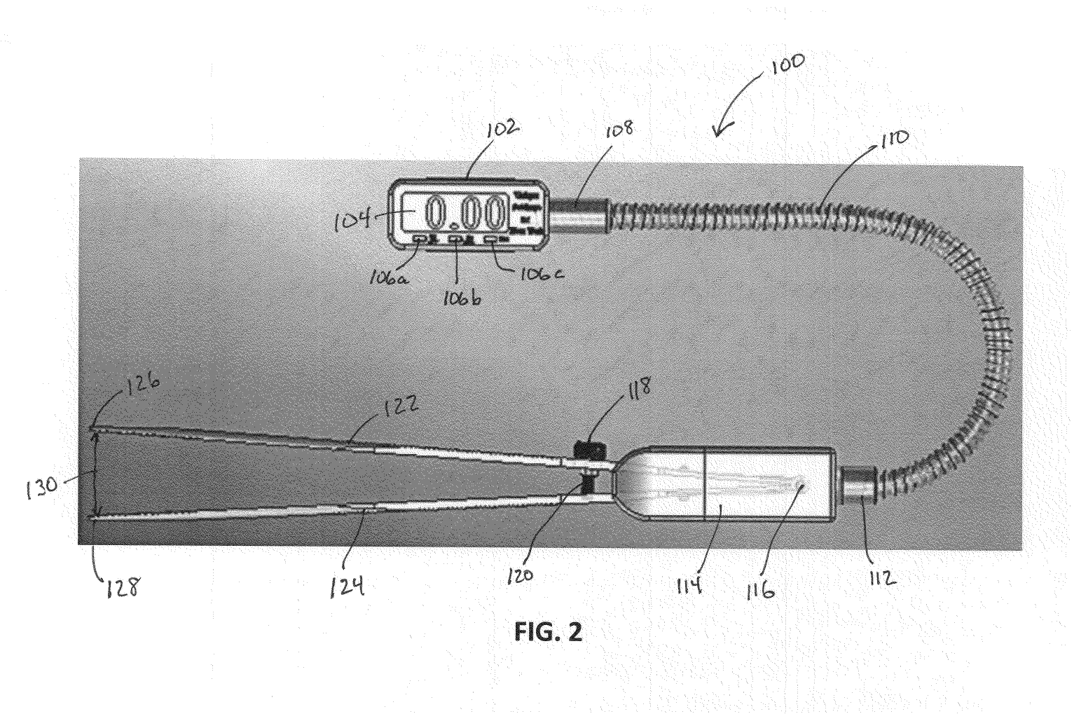

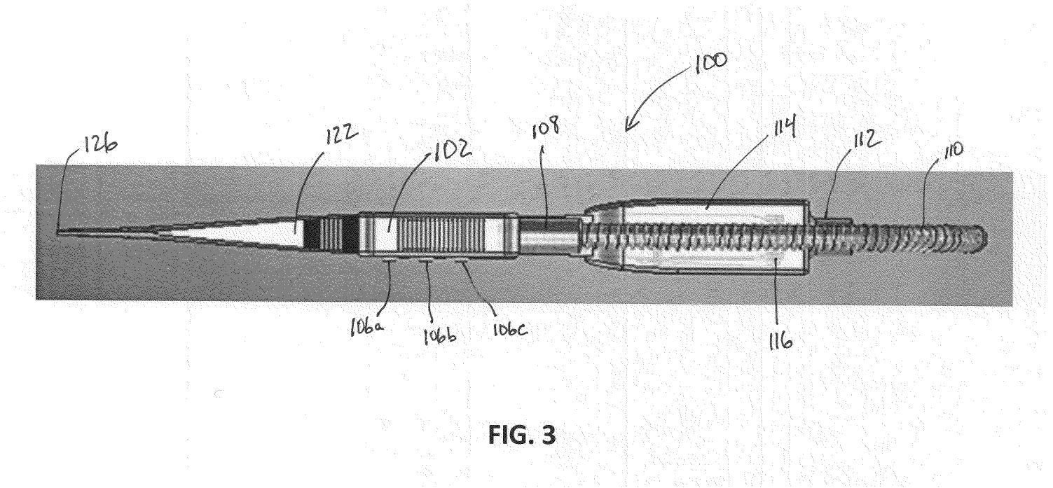

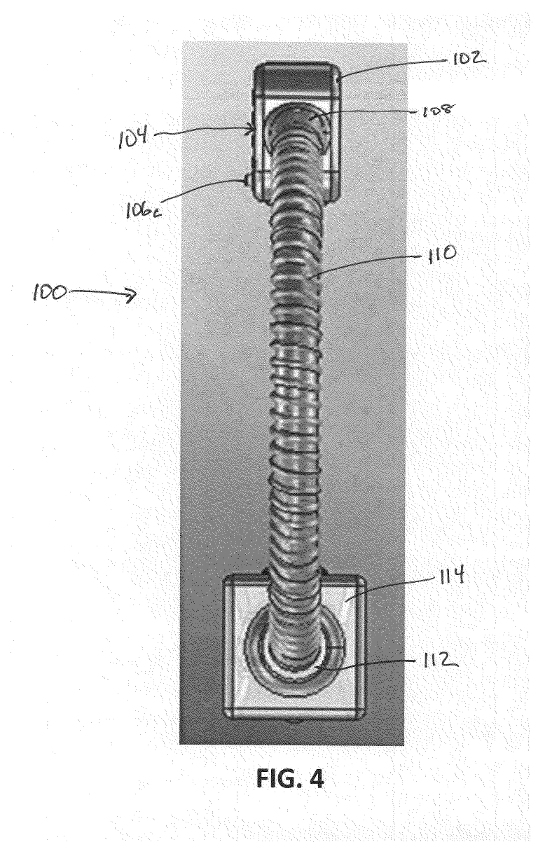

Referring first to FIGS. 1-4, shown are various views of one exemplary embodiment of measuring device 10 constructed in accordance with this invention. As will be described in more detail below, the measuring apparatus or device 10 disclosed herein includes display housing 102 including digital display 104 and display control buttons 106a, b, c. Measuring apparatus 100 further includes a pair of rigid tweezer blades 122, 124 having at a distal end thereof blade tips 126, 128 which form measuring region 130. Rigid tweezer blades 122, 124 come together at a proximal end thereof to form tweezer hinge 116. The blades 122, 124 are maintained spaced apart a predetermined distance as controlled by spacer 118, which may be controlled via spring 120. The proximal end of blades 122, 124 may optionally be housed with housing 114.

Display housing 102 and tweezer housing 114 are preferably electrically connected via connecting tube 110. Tube 110 is preferably flexible for ease of use of the measuring apparatus 100. Connectors 108, 112 are provided to interconnect tube 110 to each of the display housing 102 and tweezer housing 114, respectively. The tweezers preferably comprise a pair of spring loaded blades 122, 124 which are attached at one end 116. The other ends 126, 128 may be in the form of straight edges, points, or other suitable arrangements. These ends 126, 128 are normally held apart by the spring action of the blades. Upon the exterior surface of each blade there is preferably a roughened surface to permit a secure hold on the blades during use. When the blades are pressed together, it will be seen that the spring or spacer 118 serves as a stop to prevent the points from coming completely together. This spring or spacer 118 also provides a bearing surface for the blades such that the pressure applied to the blades is evenly distributed so that the blades do not tilt and bend.

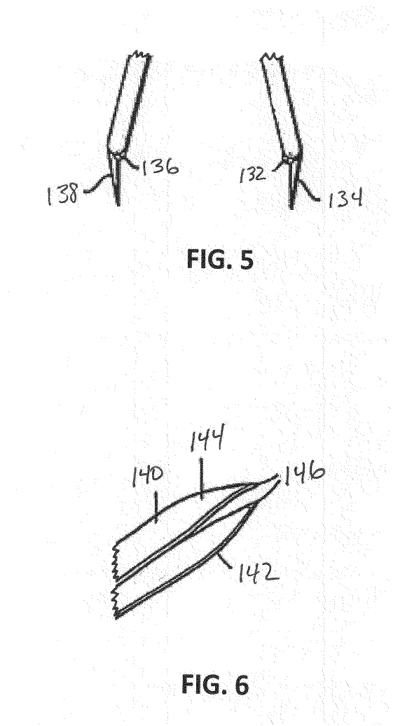

Referring next to FIGS. 5-9, shown are a front elevational view of tweezer blade tips according to a first, second, third, fourth and fifth alternative embodiments for the tweezer blades for use with the measuring device in accordance with alternate embodiments of the present invention. Referring first to FIG. 5, tweezer blades 132, 136 are shown having pair of tip 134, 138, respectively, extending outwardly from a distal end of blades 132, 136. In this embodiment, tips 134, 138 are preferably configured such that they are substantially parallel during measurement of the gemstone or other object so as to provide improved accuracy. Turning to FIG. 6, shown is a side view of another type of tweezer component 140 that may be used with the measuring device 100 in accordance with the present invention. FIG. 6 depicts a pointed tweezer component 140 where the tips of the tweezer blades 142, 144 are pointed 146. Tweezer components 142, 144 preferably have mechanisms for attaching and detaching from the tweezer housing 114 such that they are detachably engaged with housing 114 at hinge connection 116.

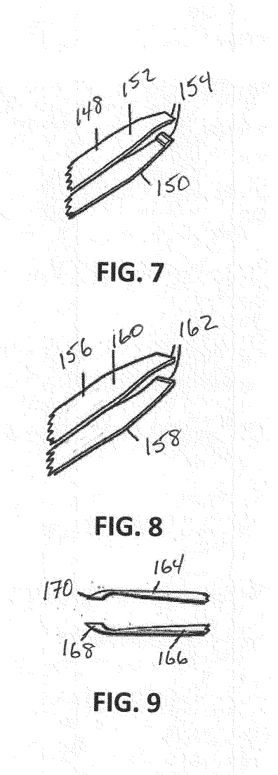

Referring to FIG. 7, shown is a side view of yet another type of tweezer component 148 that may be used with the measuring device 100 in accordance with the present invention. FIG. 7 depicts a blunt tip tweezer component 148 where the tips of the tweezer blades 150, 152 are blunted 154. Tweezer components 150, 152 preferably have mechanisms for attaching and detaching from the tweezer housing 114 such that they are detachably engaged with housing 114 at hinge connection 116. Turning next to FIG. 8, shown is a side view of still another type of tweezer component 156 that may be used with the measuring device 100 in accordance with the present invention. FIG. 8 depicts a pointed tweezer component 156 where the tips of the tweezer blades 158, 160 form a diagonal or slant edge 162. Tweezer components 158, 160 preferably have mechanisms for attaching and detaching from the tweezer housing 114 such that they are detachably engaged with housing 114 at hinge connection 116. Referring now to FIG. 9, shown is a side view of yet another type of tweezer component that may be used with the measuring device 100 in accordance with the present invention. Specifically, FIG. 9 depicts tweezer components 164, 166 where the tips of the tweezer blades 164, 166 are formed of a curved edge capped with a flat portion 168, 170. Tweezer components 164, 166 preferably have mechanisms for attaching and detaching from the tweezer housing 114 such that they are detachably engaged with housing 114 at hinge connection 116.

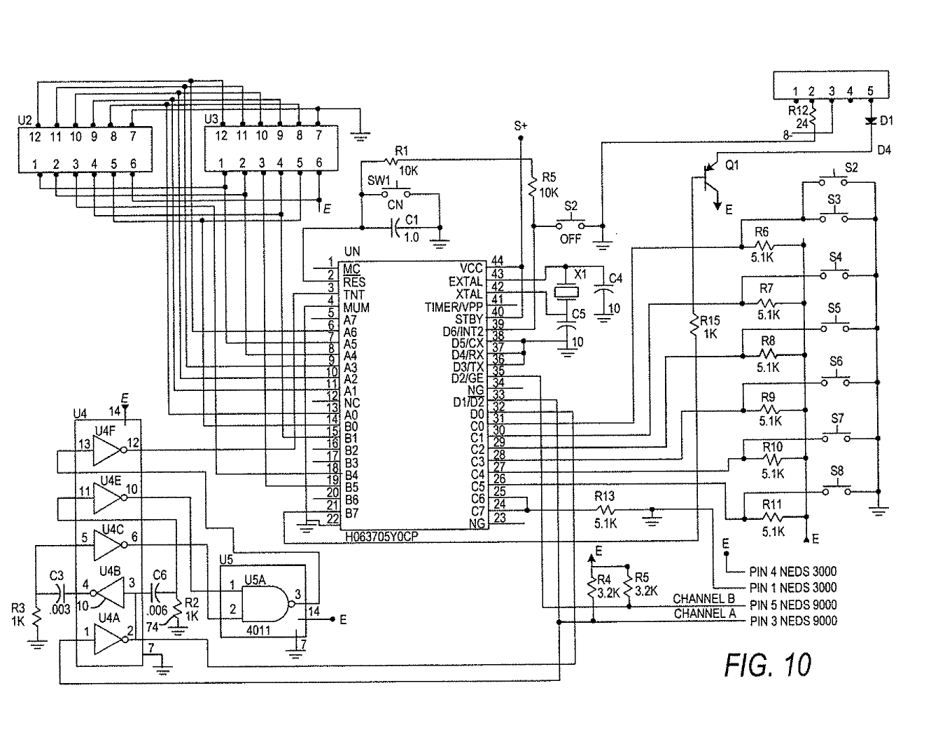

FIG. 10 shows a schematic diagram of exemplary circuitry for use in the handheld measurement device of the present invention. As shown via the schematic diagram of FIG. 10, a printed circuit board may be mounted within the housing that contains the necessary circuitry for operation of the measuring device. Preferably, a microprocessor receives from the sensing assembly inputs which represent movement of tweezer blades, as well as input from display control buttons and provides outputs to display.

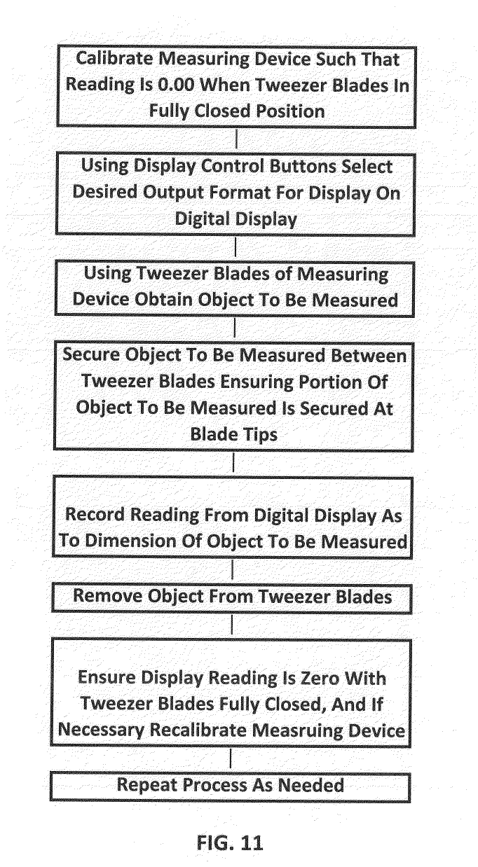

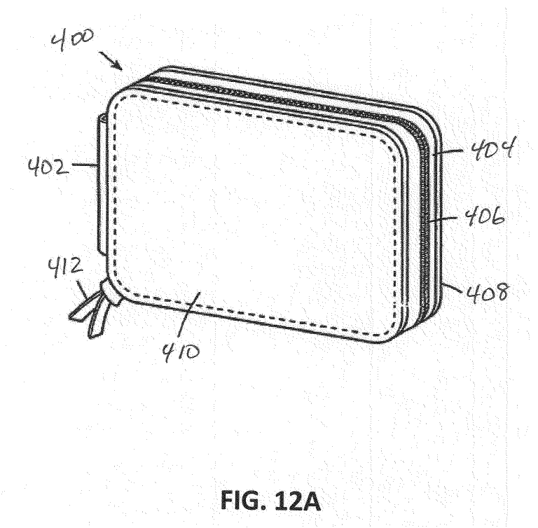

Also in accordance with the present invention, provided is a method for measuring any of a variety of dimensions of small objects such as gemstones. Specifically, FIG. 11 shows a flow chart illustrating an exemplary operation of the handheld measurement device of the present invention wherein the method comprises the steps of calibrating the measuring device, locating an object to be measured, securing the object at a distal end of a pair of blade members, and reading a displayed dimension of the object on a display device. Initially, measuring device 100 must be calibrated such that a zero reading is displayed when the tweezer blades 122, 124 are in the fully closed position. Then, using the display control buttons 106 a,b,c, the desired output is selected for display on the digital display 104. Using the tweezer blades 122, 124 of the measuring device 100, an object to be measured is obtained. The object is secured between the tweezer blades 122, 124 ensuring that the portion to be measured is secured at the blade tips 126, 128. With the object secured between the blade tips 126, 128, the digital display 104 will display the dimension of the object secured between blade tips 126, 128. This dimension is manually recorded as desired. Optionally, measuring device 100 may include programming to allow for automated recording and storage of the dimension of any number of objects being measured. Once recorded, the object is removed from the tweezer blades 122, 124. The measuring device is then checked to ensure the proper calibration such that a zero reading is displayed when the tweezer blades are in a closed position. If necessary, the device 100 is recalibrated. The process is then repeated as many times as is necessary.

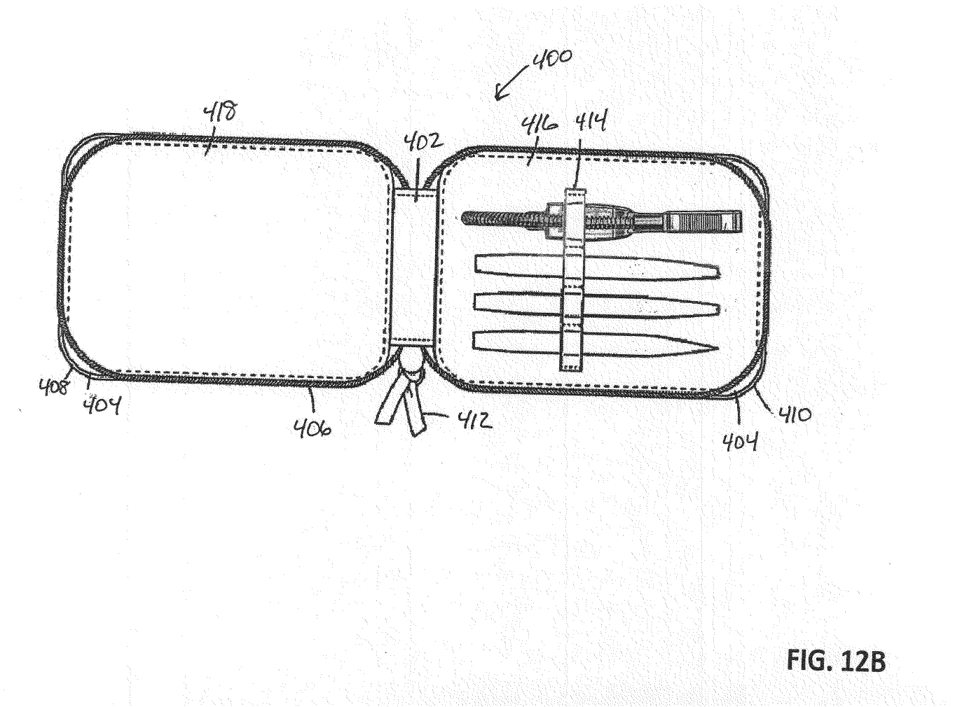

Referring last to FIGS. 12A and 12B, shown is a perspective view of a measuring device kit 400 in accordance with an exemplary embodiment of the present invention. As shown, the kit 400 provides a case having a zippered closure 406, which has a sidewall 404 preferably made from leather, plastic, cloth, etc. The case is preferably closed by a zipper 406, with a zipper handle 412 that facilitates opening and closing of the zipper 406. In one embodiment, the case 400 preferably comprises a top half 410 and a bottom half 408 that are foldable on top of each other by folding the top half 410 over the bottom half 408 on seam 402. The case 400 may be closed by zipper 406 using zipper handle 412. Internal to the case 400 there are preferably inserts or compartments for maintaining tweezer measuring device 100, along with any number of additional replacement tweezer blades 420, which may be affixed to the inner surface 416 of top half 410 or the inner surface 418 of bottom half 408. The measuring device 400 and tweezer blade components 420 may be affixed to inner surface 416 or 418 by elasticized loops 414 or other similar securing mechanism.

### REFERENCE NUMERALS

measuring apparatus 100
display housing 102
digital display 104
display control buttons 106a, b, c
connector 108
flexible connecting tube 110
connector 112
tweezer housing 114
tweezer hinge 116
spring or spacer 118
spring 120
rigid blades 122, 124
blade tips 126, 128
measuring region 130
tweezer blades 132
pointed straight tip 134
tweezer blades 136
pointed straight tip 138
tweezer component 140
tweezer blades 142
tweezer blades 144
pointed curved tips 146
tweezer component 148
tweezer blades 150
tweezer blades 152
blunted tips 154
tweezer component 156
tweezer blades 158
tweezer blades 160
diagonal slanted tips 162
tweezer blades 164
tweezer blades 166
curved tip capped with flat portion 168
curved tip capped with flat portion 170
circuitry 200
flow diagram 300
case 400
seam 402
case sidewall 404
zippered closure 406
case bottom half 408
case top half 410
zipper handle 412
elastic loops 414
inner surface 416
inner surface 418

In the claims, means or step-plus-function clauses are intended to cover the structures described or suggested herein as performing the recited function and not only structural equivalents but also equivalent structures. Thus, for example, although a nail, a screw, and a bolt may not be structural equivalents in that a nail relies on friction between a wooden part and a cylindrical surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures.

Having described at least one of the preferred embodiments of the present invention with reference to the accompanying drawings, it is to be understood that such embodiments are merely exemplary and that the invention is not limited to those precise embodiments, and that various changes, modifications, and adaptations may be effected therein by one skilled in the art without departing from the scope or spirit of the invention as defined in the appended claims. The scope of the invention, therefore, shall be defined solely by the following claims. Further, it will be apparent to those of skill in the art that numerous changes may be made in such details without departing from the spirit and the principles of the invention. It should be appreciated that the present invention is capable of being embodied in other forms without departing from its essential characteristics.

What is claimed is:

1. A handheld digital measuring device, said device comprising:
a housing having first and second blade members extending therefrom, said first and second blade members each having proximal and distal ends thereof, and said first blade member being opposingly movable relative to said second blade member;
a sensor for sensing movement of said blade members and providing a signal representative of the opposing movement of said blades members to provide a dimensional measurement of an object positioned between said distal ends of said first and second blade members; and
a display device for receiving said signal from said sensor for providing a visual display of said dimensional measurement of said object.

2. The measuring device according to claim 1, wherein said display device includes a keypad for selecting a format for display of said dimensional measurement.

3. The measuring device according to claim 1, wherein said display device includes a keypad for resetting said display.

4. The measuring device according to claim 1, wherein said display device includes a keypad for calibrating said device.

5. The measuring device according to claim 1, wherein said display device includes means for resetting said dimensional measurements at any selected position of said blade members.

6. The measuring device according to claim 1, further comprising a spring-loaded pneumatic mechanism coupled between said housing and said distal end of said blade members for urging said first and second blade members apart.

7. The measuring device according to claim 1, wherein said distal end of said blade member comprises an angled portion configured such that said angled portion of said first blade is substantially parallel with said angled portion of said second blade during use of said measuring device.

8. The measuring device according to claim 1, wherein the blade members comprise at least one of a slant tip, blunt tip, or pointed tip tweezer

9. A handheld digital measuring instrument having a digital display, said instrument comprising:
a pair of relatively elongated blade members extending from a housing, said first and second blade members each having proximal and distal ends thereof, and said first and second blade members being rotatably movable about an axis;
a sensor for measuring a distance between said blade members at a defined point along said blade members, said sensor capable of providing a signal representative of said distance to provide a dimensional measurement of an object positioned between said distal ends of said blade members; and
a display device for receiving said signal from said sensor for providing a display of said dimensional measurement.

10. The measuring device according to claim 9, wherein said display device includes a keypad for selecting a format for display of said dimensional measurement.

11. The measuring device according to claim 9, wherein said display device includes a keypad for resetting said display.

12. The measuring device according to claim 9, wherein said display device includes a keypad for calibrating said device.

13. The measuring device according to claim 9, wherein said display device includes means for resetting said dimensional measurements at any selected position of said blade members.

14. The measuring device according to claim 9, further comprising a spring-loaded pneumatic mechanism coupled between said housing and said distal end of said blade members for urging said first and second blade members apart.

15. The measuring device according to claim 9, wherein said distal end of said blade member comprises an angled portion configured such that said angled portion of said first blade is substantially parallel with said angled portion of said second blade during use of said measuring device.

16. The measuring device according to claim 9, wherein the blade members comprise at least one of a slant tip, blunt tip, or pointed tip tweezer.

17. A kit for providing a handheld digital measuring device, wherein said kit comprises:
a case body having a closure mechanism;
at least one handheld measuring device having a display and a housing for receiving a pair of blade members; and
at least one pair of interchangeable blade members that may be interchangeably and detachably engaged with said housing.

18. The kit according to claim 17, wherein the closure mechanism is a zipper closure.

19. The kit according to claim 17, wherein the measuring device and blade members are removably secured to the inside of the case by elastic loops.

20. The kit according to claim 17, wherein the blade members comprise at least one of a slant tip, blunt tip, or pointed tip tweezer.
